(19) United States
(12) Patent Application Publication
(10) Pub. No.: US 2023/0052520 A1
(43) Pub. Date: Feb. 16, 2023

(54) ELECTROPORATION ABLATION FOR THE TREATMENT OF TYPE II DIABETES

(71) Applicant: Medtronic, Inc., Minneapolis, MN (US)

(72) Inventors: Lars M. Mattison, Blaine, MN (US); Steven J. Fraasch, Maple Grove, MN (US); Steven V. Ramberg, North Oaks, MN (US); Brian J. Ross, Maple Grove, MN (US)

(21) Appl. No.: 17/818,819

(22) Filed: Aug. 10, 2022

Related U.S. Application Data
(60) Provisional application No. 63/232,937, filed on Aug. 13, 2021.

(51) Int. Cl. A61B 18/12; A61B 18/14

(52) U.S. Cl. CPC A61B 18/1206; A61B 18/1492; A61B 2018/00345

(57) ABSTRACT

A system for hepatic nerve denervation includes a medical device and a generator in communication with the medical device. The medical device includes an elongate body having a proximal portion and a distal portion opposite the proximal portion, and a plurality of treatment electrodes coupled to the distal portion. The distal portion is configured to be in contact with an area of target tissue. The area of target tissue is an area of tissue within the hepatic artery. The generator is configured to generate and deliver at least one pulse train of energy to the plurality of treatment electrodes to ablate the area of target tissue.

### CROSS-REFERENCE TO RELATED APPLICATION

This application claims the benefit of U.S. Provisional Patent Application No. 63/232,937, filed 13 Aug. 2021, the entire contents of which is incorporated herein by reference.

### TECHNICAL FIELD

The present technology is generally related to systems, methods, and related devices for hepatic nerve denervation.

### BACKGROUND

Type II diabetes (formerly called non-insulin-dependent, or adult-onset diabetes) results from the body of a living organism's ineffective use of insulin, and is largely the result of excess body weight and/or physical activity. The majority of people with diabetes around the world have Type II diabetes. Recent publications have shown a potential link between nerve activity and an increased level of blood glucose.

### SUMMARY

The techniques of this disclosure generally relate to a device, system, and related methods thereof, for hepatic nerve denervation. For instance, a device may be configured to deliver electrical energy to cause electroporation ablation to treat Type II diabetes.

In one aspect, a system for hepatic nerve denervation includes a medical device and a generator in communication with the medical device. The medical device includes an elongate body having a proximal portion and a distal portion opposite the proximal portion, and a plurality of treatment electrodes coupled to the distal portion. The distal portion is configured to be in contact with an area of target tissue. The area of target tissue is an area of tissue within the hepatic artery. The generator is configured to generate and deliver at least one pulse train of energy to the plurality of treatment electrodes to ablate the area of target tissue.

In another aspect, the plurality of treatment of electrodes are arranged in a plurality of pairs, each pair of treatment electrodes having a polarity that is different than a polarity of an adjacent treatment electrode pair.

In another aspect, the at least one pulse train of energy is delivered in one of a bipolar mode and a monopolar mode from the generator.

In another aspect, the system further includes at least one sensing electrode coupled to the distal portion of the elongate body, the at least one sensing electrode is configured to continuously measure and record a plurality of glucose measurements within the area of target tissue, the plurality of glucose measurements are indicative of a measured level of glucose uptake within the area of target tissue.

In another aspect, the system further includes a control unit in communication with the medical device. At least one electrode pair of the plurality of treatment electrodes is configured to record at least one of an EMG signal and a compound action potential (CAP) signal and transmit the at least one of the EMG signal and CAP signal to the control unit.

In another aspect, the control unit includes processing circuitry. The processing circuitry is configured to: establish at least one of a reference electromyogram and baseline CAP recording of the area of target tissue prior to the delivery of the at least one pulse train of energy; obtain at least one of an electromyogram and second CAP recording of the area of target tissue following the delivery of the at least one pulse train of energy; at least one of: compare the obtained electromyogram to the reference electromyogram, and compare the second CAP recording to the baseline CAP recording; and generate an alert based at least in part on the comparison.

In another aspect, the processing circuitry is further configured to determine whether denervation has occurred in the area of target tissue based on the comparison of the obtained electromyogram and the reference electromyogram or the comparison of the second CAP recording to the baseline CAP recording.

In another aspect, the processing circuitry is further configured to discontinue the delivery of the at least one pulse train of energy from the generator when it is determined that denervation has occurred in the area of target tissue.

In another aspect, the at least one pulse train of energy is delivered in a biphasic mode from the plurality of treatment electrodes.

In another aspect, the processing circuitry is further configured to determine whether denervation has occurred in the area of target tissue based on the measured level of glucose uptake within the area of target tissue.

In yet another aspect, a system for hepatic nerve denervation includes: a medical device and a generator. The medical device includes an elongate body and a plurality of treatment electrodes. The elongate body has a proximal portion and a distal portion opposite the proximal portion. The distal portion is configured to be in contact with an area of target tissue. The area of target tissue is an area of tissue within the hepatic artery. The plurality of treatment electrodes are coupled to and disposed about the distal portion. The generator is in electrical communication with the medical device and is configured to generate and deliver at least one pulse train of energy to the plurality of treatment electrodes to ablate the area of target tissue.

In another aspect, the plurality of treatment of electrodes are arranged in a plurality of pairs, each pair of treatment electrodes having a polarity that is different than a polarity of an adjacent treatment electrode pair.

In another aspect, the at least one pulse train of energy is delivered in one of a bipolar mode and a monopolar mode from the generator.

In another aspect, the system further includes at least one sensing electrode coupled to the elongate body, the at least one sensing electrode is configured to continuously record a plurality of glucose measurements within the area of target tissue, the plurality of glucose measurements is indicative of a measured level of glucose uptake within the area of target tissue.

In another aspect, a control unit is in communication with the medical device. At least one electrode of the plurality of treatment electrodes is configured to record at least one of an EMG signal and a compound action potential (CAP) signal and transmit the at least one of the EMG signal and CAP signal to the control unit.

In another aspect, the control unit includes processing circuitry. The processing circuitry is configured to: establish at least one of a reference electromyogram and baseline CAP recording of the area of target tissue prior to the delivery of the at least one pulse train of energy; obtain at least one of an electromyogram and second CAP recording of the area of target tissue following the delivery of the at least one pulse train of energy; at least one of: compare the obtained electromyogram to the reference electromyogram, and compare the second CAP recording to the baseline CAP recording; and generate an alert based at least in part on the comparison.

In another aspect, the processing circuitry is further configured to determine whether denervation has occurred in the area of target tissue based on the comparison of the obtained electromyogram and the reference electromyogram or the comparison of the second CAP recording and the baseline CAP recording.

In another aspect, the processing circuitry is further configured to discontinue the delivery of the at least one pulse train of energy from the generator when it is determined that denervation has occurred in the area of target tissue.

In another aspect, the processing circuitry is further configured to determine whether denervation has occurred in the area of target tissue based on the measured level of glucose uptake within the area of target tissue.

In yet another aspect, a method of denervating a hepatic nerve includes: positioning a medical device proximate to an area of target tissue within the hepatic artery, the medical device including an elongate body with a proximal portion and a distal portion opposite the proximal portion; measuring at least one EMG signal at the area of target tissue and generating a reference electromyogram, the at least one EMG signal being measured by a plurality of electrodes disposed on the distal portion of the elongate body; delivering pulsed electric field (PEF) energy to plurality of electrodes to treat the area of target tissue, the delivered PEF energy being generated by an energy generator. The method further includes at least one selected from the group consisting of: measuring a level of glucose uptake at the area of target tissue following the delivery of PEF energy, and measuring at least one EMG signal at the area of target tissue following the delivery of PEF energy and generating a second electromyogram. The method further includes determining an efficacy of the delivery of PEF energy based on at least one selected from the group consisting of the measured level of glucose uptake and a comparison of the second electromyogram to the reference electromyogram.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present disclosure, and the attendant advantages and features thereof, will be more readily understood by reference to the following detailed description when considered in conjunction with the accompanying drawings wherein:

FIG. 1 shows an exemplary system for hepatic nerve denervation constructed in accordance with the principles of the present disclosure.

FIG. 2 shows a distal portion of an exemplary medical device for use with the system of FIG. 1, the distal portion including a plurality of treatment electrodes and a glucose sensor.

FIG. 3 shows a 3-electrode potentiostat configuration of the glucose sensor of FIG. 2.

FIGS. 4 and 5 show the medical device of the present disclosure positioned within the gastrointestinal system.

FIG. 6 shows a flow chart describing a method in accordance with the principles of the present disclosure.

FIG. 7 is a conceptual diagram illustrating example energy vectoring, in accordance with one or more aspects of this disclosure.

### DETAILED DESCRIPTION

The present application provides methods and systems for diagnosing and/or treating undesirable physiological or anatomical tissue regions, such as those contributing to the production of glucose in the liver.

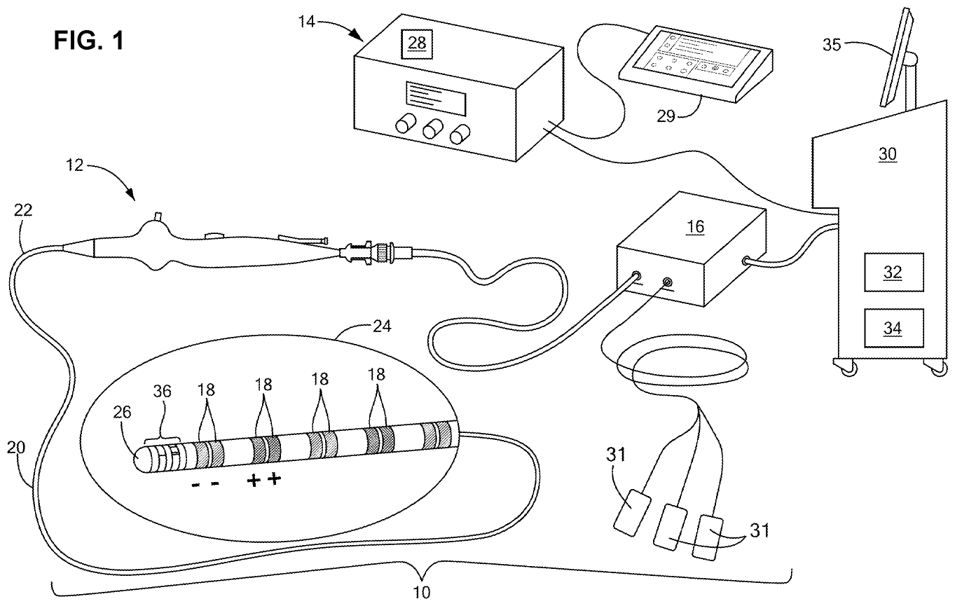

Referring now to the drawing figures in which like reference designations refer to like elements, an embodiment of a medical system constructed in accordance with principles of the present disclosure is shown in FIG. 1 and generally designated as “10.” The system 10 generally includes a medical device 12 that may be coupled directly to an energy supply, for example, an electroporation energy generator 14 including an energy control, delivering and monitoring system, or indirectly through a device electrode distribution system 16 (which may also be referred to herein as a catheter electrode distribution system or CEDS). A remote controller 15 may further be included in communication with the generator 14 for operating and controlling the various functions of the generator 14. As discussed in more detail below, the medical device 12 may generally include one or more diagnostic or treatment regions for energetic, therapeutic and/or investigatory interaction between the medical device 12 and a treatment site. The treatment region(s) may deliver, for example, trains of high voltage in very short duration electrical pulses to injure an area of tissue in proximity to the treatment region(s) by the mechanism of electroporation. As a non-limiting example, the treatment region(s) may include a plurality of electrodes 18 configured to deliver electroporation energy to a tissue area in proximity to the electrodes 18.

Referring now to FIG. 1, the medical device 12 may be a catheter such as, for example, an electroporation catheter as shown in FIG. 1, and may be adapted for use with the generator 14. The generator 14 may provide electrical pulses to the device 12 to perform the electroporation procedure to an area of target tissue such as, for example, an area of tissue within the hepatic artery or other surrounding tissues within the body such as, for example, splanchnic nerve tissue, vagal nerve tissue, and organs or tissue surrounding the liver. “Electroporation” utilizes high amplitude pulses to effectuate a physiological modification (i.e., permeabilization) of the cells to which the energy is applied. Such pulses may preferably be short (e.g., nanosecond, microsecond, or millisecond pulse width) in order to allow application of high voltage, high current (for example, 20 or more amps) without long duration of electrical current flow that results in significant tissue heating and muscle stimulation. In particular, the pulsed energy induces the formation of microscopic pores or openings in the cell membrane. The use of electroporation energy reduces the possibility of collateral damage to tissue structures surrounding the area of target tissue, depending on the applied pulse parameters. High voltage targeted delivery across the nerves allows for focused energy delivery while also greatly minimizing damage to collateral structures, such as the arteries, as well as reducing the potential for adverse events such as blood coagulum. Also, depending upon the characteristics of the electrical pulses, an electroporated cell can survive electroporation (i.e., “reversible electroporation”) or die (i.e., irreversible electroporation, “IEP”). Reversible electroporation may be used to transfer agents, including large molecules, into targeted cells for various purposes, including alteration of the nerves which innervate the liver, resulting in reduced production and storage of glucose.

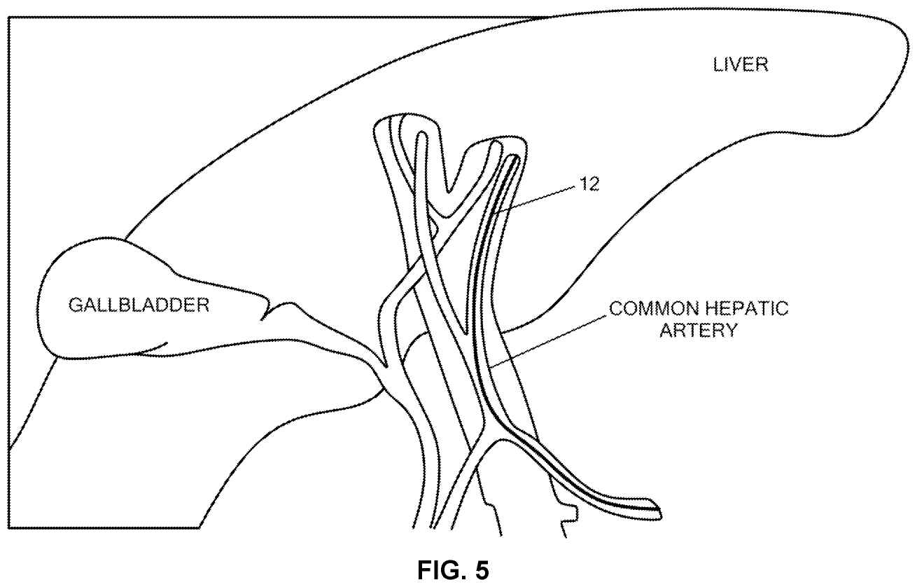

The device 12 may include an elongate body 20 passable through a patient's vasculature and/or positionable proximate to an area of target tissue for diagnosis or treatment, such as a catheter sheath or intravascular introducer. The elongate body 20 may define a proximal portion 22 and a distal portion 24, and may further include one or more lumens disposed within the elongate body 20 thereby providing mechanical, electrical, and/or fluid communication between the proximal portion of the elongate body 20 and the distal portion 24 of the elongate body 20. The distal portion 24 may define a distal tip 26 portion and may generally define the one or more treatment region(s) of the medical device 12 that are operable to monitor, diagnose, and/or treat a portion of a patient. The treatment region(s) may have a variety of configurations to facilitate such operation. In the case of purely bipolar pulsed field delivery, distal portion 24 includes electrodes that form the bipolar configuration for energy delivery. In one embodiment at least a portion of the distal portion 24 or the distal tip 26 may be coated in a magnetic material such as iron oxide, and the like, to assist clinicians with navigation and placement of the device 12 within the patient's vasculature. Further, the elongate body 20 may be rigid and/or flexible to facilitate the navigation of the device 12 within the patient's body. In one embodiment, the distal portion 24 of the elongate body 20 is flexible to allow for more desirable position proximate to the area of target tissue within the hepatic artery. As shown in FIGS. 4 and 5, to access the area of target tissue, the device 12 may be inserted through the subclavian artery or femoral artery, past the iliac artery and towards the celiac trunk, and then advanced to the hepatic artery.

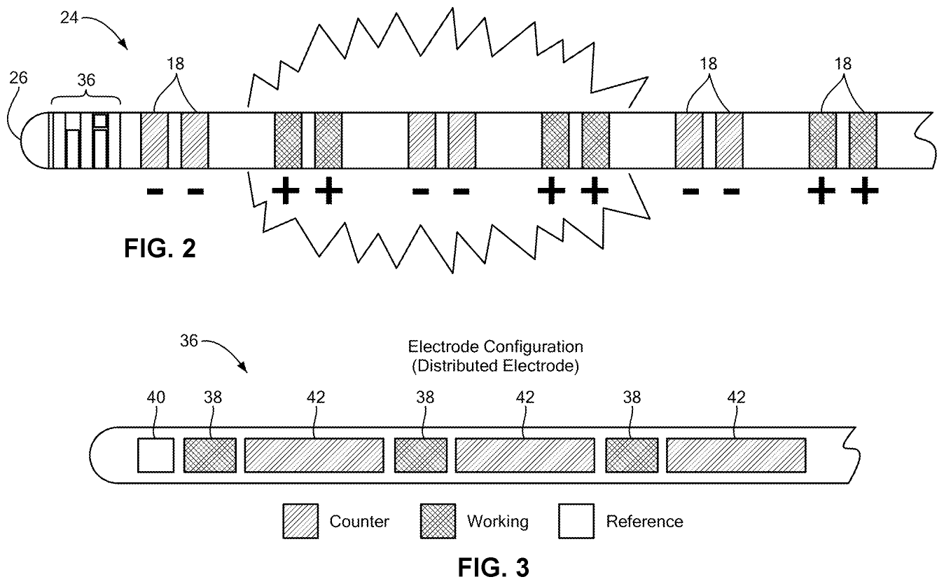

Now referring to FIG. 2, the elongate body 20 may include a plurality of treatment electrodes 18 at the distal portion 24 for delivering the electroporation energy to the area of target tissue. In particular, each treatment electrode 18 is configured such that high voltage PEF energy may be delivered from the generator 14, through the treatment electrode 18, and to the target tissue, in order to perform reversible and/or irreversible electroporation and/or ablation and, consequently, denervation of the hepatic artery. As shown in FIG. 2, the treatment electrodes 18 may be arranged in alternating pairs of positively charged electrodes and negatively charged electrodes along the distal portion 24. Although each individual electrode 18 in each pair of electrodes is shown as being uniformly spaced apart from a second electrode 18 in each pair, it is to be understood that the pairs of electrodes are not limited to this particular spacing. In other words, the spacing between adjacent electrodes 18 in a first pair of electrodes may be different from the spacing between adjacent electrodes 18 in a second pair of electrodes. Similarly, although each pair of electrodes 18 is uniformly spaced apart from an adjacent pair of electrodes 18, it is to be understood that the spacing between electrode pairs may be such that the distance between a first electrode pair and a second electrode pair may be difference than the spacing between a third electrode pair and a fourth electrode pair.

In one embodiment, the generator 14 may include processing circuitry 28 having a processor in communication with one or more controllers and/or memories containing software modules containing instructions or algorithms to provide for the automated operation and performance of the features, sequences, calculations, or procedures described herein. The processing circuitry 28 of the generator 14 may be configured to monitor, record or otherwise convey measurements or conditions within the device 12 or the ambient environment at the distal portion of the device 12, additional measurements may be made through connections to the multi-electrode catheter including for example temperature, electrode-tissue interface impedance, delivered charge, current, power, voltage, work, or the like in the generator 14 and/or the device 12. Neutral electrode patient ground patches (not pictured) may be employed to evaluate the desired bipolar electrical path impedance, as well as monitor and alert the operator upon detection of inappropriate and/or unsafe conditions. which include, for example, improper (either excessive or inadequate) delivery of charge, current, power, voltage and work performed by the treatment electrodes 18; improper and/or excessive temperatures of the treatment electrodes 18; improper electrode-tissue interface impedances; improper and/or inadvertent electrical connection to the patient prior to delivery of high voltage energy by delivering one or more low voltage test pulses to evaluate the integrity of the tissue electrical path. Further, the generator 14 may further include or be in electrical communication with one or more other system components, such as one or more user input devices 27.

The generator 14 may be an electrical current or pulse generator having a plurality of output channels, with each channel coupled to an individual electrode of the treatment electrodes 18 or multiple electrodes of the treatment electrodes 18 of the device 12. The generator 14 may be operable in one or more modes of operation, including for example: (i) bipolar energy delivery between at least two treatment electrodes 18 or electrically-conductive portions of the medical device 12 within a patient's body, (ii) monopolar or unipolar energy delivery to one or more of the electrodes 18 or electrically-conductive portions on the device 12 within a patient's body and through either a second device within the body (not shown) or a patient return or ground electrode (not shown) spaced apart from the treatment electrodes xx of the device 12, such as on a patient's skin or on an auxiliary device positioned within the patient away from the device 12, for example, and (iii) a combination of the monopolar and bipolar modes.

The generator 14 may be configured and programmed to generate and deliver pulsed, high voltage electric fields appropriate for achieving desired pulsed, high voltage ablation (or pulsed field ablation), to the medical device 12. As a point of reference, the pulsed, high voltage, non-radiofrequency, ablation effects of the present disclosure are distinguishable from DC current ablation, as well as thermally-induced ablation attendant with conventional RF techniques. For example, the pulse trains generated by generator 14 are delivered at a frequency less than 10 kHz, and in an exemplary configuration, 500 Hz to 10 kHz. The pulsed-field energy in accordance with the present disclosure is sufficient to induce cell death for purposes of damaging or killing the nerves which innervate the liver, with the goal of reducing sympathetic tone to the metabolic organs.

The generator 14 may generate and deliver therapeutic biphasic, bipolar pulses having a preprogrammed pattern and duty cycle through the treatment electrodes 18 to localize the electric field to the targeted area while minimizing neuromuscular stimulation that can be caused by unipolar deliveries. For example, each pulse cycle may include an applied voltage amplitude A, a pulse width B (in μs), an inter-phase delay C (in μs), an inter-pulse delay D (in μs), and a pulse cycle length E. In an exemplary configuration, the pulse width B may be between the range of approximately 100 ns to 500 μs, the inter-phase delay C may be between the range of approximately 5 μs to 100 μs, the inter-pulse delay D may be between the range of approximately 100 μs to 2000 μs, the pulse train may include 10-500 pulses, and the applied voltage may be approximately 500-4000 V. The pulse trains may produce lesions in hepatic artery tissue approximately 5 mm to 10 mm deep. Increased voltage may correspondingly increase the lesion depth.

The pulsed field of energy may be delivered in a bipolar fashion, between odd and even electrodes, in monophasic or biphasic pulses. The application of biphasic electrical pulses may produce unexpectedly beneficial results in the context of hepatic tissue ablation. With biphasic electroporation pulses, the direction of the pulses completing one cycle alternates in a few microseconds. As a result, the cells to which the biphasic electrical pulses are applied undergo alternation of electrical field bias. Changing the direction of bias reduces prolonged post-ablation depolarization and/or ion charging.

The treatment electrodes 18 may be configured to monitor or record electromyography (“EMG”) measurements of the hepatic artery. An electromyograph detects the electrical action potential generated by muscle cells when these cells are both mechanically active and at rest. Once the device 12 is positioned proximate to the area of target tissue and the treatment electrodes are in contact with the target tissue, electrical signals may be taken upon contraction of the muscle (such as in response to the induced excitation of the targeted tissue structure) and again during relaxation. The shape, size and frequency of the resulting muscle motor unit potentials can then be analyzed to establish a baseline or threshold value for later comparison. In cases where intramuscular EMG may be considered too invasive or unnecessary, one or more surface electrodes 31 may be used to monitor the muscle activation. As discussed above, the liver is largely responsible for both storage and manufacturing of glucose, and may be targeted for one or more treatment applications (such as electroporation to denervate the hepatic nerve area, for example). The induced response may then be used to establish or otherwise define a threshold or baseline value. Subsequent activity or physiological changes occurring in the patient during a therapeutic procedure may be compared to the baseline or threshold value and thus generate an alert and/or be used to modify one or more parameters of the delivered treatment. Although the system 10 may be used to stimulate and monitor the hepatic nerve area, it will be understood that other areas of non-target tissue and/or anatomical structures, such as other nerves or muscles, may instead be stimulated and monitored to prevent incurring damage to those non-target tissues during the delivery of treatment or ablation energy to an area of target tissue. In one embodiment, at least one electrode of the plurality of treatment electrodes 18 may be configured to record at least one EMG signal from the area of target tissue and transmit the at least one EMG signal to a control unit 30 having processing circuitry 32 and memory 34 to establish a reference electromyogram (which may also be referred to herein as an established electromyogram threshold). Once the area of target tissue has been treated with the electroporation energy, the treatment electrodes 18 are configured to once again record at least one EMG signal (indicative of biological electrical activity) and transmit the EMG signal to the control unit 30 to generate a second electromyogram that may then be compared to the reference electromyogram. Based on the comparison of the reference electromyogram and the second electromyogram, an alert may be generated by the control unit 30 and shown on a display 35 to notify the clinician as to the efficacy of the procedure, and specifically, whether denervation has occurred in the area of target tissue. The control unit 30 may then discontinue the delivery of the at least one pulse train of energy from the generator 14 when it is determined that denervation has occurred in the area of target tissue.

Additionally, the treatment electrodes 18 may be configured to record compound action potential (CAP) signal and transmit the recorded CAP signals to the control unit 30 to generate a CAP recording prior to, during, or after the treatment of target tissue with electroporation energy. For example, an electrical stimulus (e.g., a pacing pulse) is used to generate a CAP (i.e., stimulate the target nerve and measure the compound action potential as it propagates down the nerve). The electrodes 18 are configured to record a baseline CAP recording prior to the delivery of electroporation energy and a second CAP recording following the delivery of electroporation energy. The second CAP recording may then be compared to the baseline CAP recording. Based on the comparison, an alert may be generated by the control unit 30 and shown on the display 35 to notify the clinician as to the efficacy of the procedure, and specifically, whether denervation has occurred in the area of target tissue. The control unit 30 may then discontinue the delivery of the at least one pulse train of energy from the generator 14 when it is determined that denervation has occurred in the area of target tissue.

Further, in one embodiment, the generator 14 may be configured to generate and selectively deliver energy to electrodes 18 such that not all of the electrodes 18 are used for treatment. For example, as shown in FIG. 2, a pair of electrodes 18 may be used to treat tissue while surrounding proximal and distal electrodes 18 may be used to record the EMG signals. This proximal and distal positioning of electrodes 18 to record EMG measurements outside of the ablation field allows for assessment of acute denervation through application of stimulus pulses distally and EMG measurement proximally. In doing so, this allows clinicians to assess nerve function acutely.

Referring now to FIGS. 1-3, the device 12 may include one or more sensors 36 coupled to the distal portion 24 of the elongate body 20 for detecting pressure, temperature, electrical impedance, glucose production, or other system and/or environmental parameters (for example, the surface temperature of the target tissue). The one or more sensors 36 may be of any configuration such as, for example, ring sensors, distal tip sensors, round sensors, or balloon sensors, and may be in communication with the control unit 30.

In one exemplary embodiment, the sensors 36 are blood glucose sensors disposed on the distal portion 24 proximate to the distal tip 26, and are configured to periodically and/or continuously stimulate tissue, sense, and/or record electrochemical data, and electrical and/or compound action potential signals from within the smooth muscle tissue of the hepatic artery. This allows for the monitoring of nerve function before and after the ablation procedure and the opportunity to evaluate the efficacy of the ablation. In particular, the glucose sensors 36 allow for acute detection during the procedure to measure glucose uptake. During the procedure, glucose challenges can be bolused into the patient. The glucose sensors 36 allow for both baseline assessment prior to treatment (initial bolus) and post treatment (subsequent bolus). Net glucose can then be assessed to determine acute procedural success.

The signals are then transmitted to the control unit 30 and used to generate at least one glucose measurement. The control unit 30 may then correlate the glucose measurements with a quality of lesion formation in the area of target tissue. For example, a high measured level of glucose may indicate a lower quality lesion, whereas a low measured level of glucose may indicate a higher quality of lesion. Lesion quality is based, in part, on the size and depth of the lesion. As such, higher quality lesions may result in larger and/or deeper lesions in the area of target tissue which may be desirable when ablating tissue within the hepatic artery to limit glucose production. Additionally, if the glucose sensor 36 detects a low level of glucose production after treating the area of target tissue with electroporation energy, the electroporation energy application may be stopped either automatically by the control unit 30 or manually by the user. Although not described in detail herein, the device 12 may also be positioned within the hepatic artery under fluoroscopic guidance in which the treatment electrodes 18 and/or glucose sensors 36 may be configured to stimulate the area of target tissue and record the electrical action potential signals from within the smooth muscle tissue in response to the stimulus.

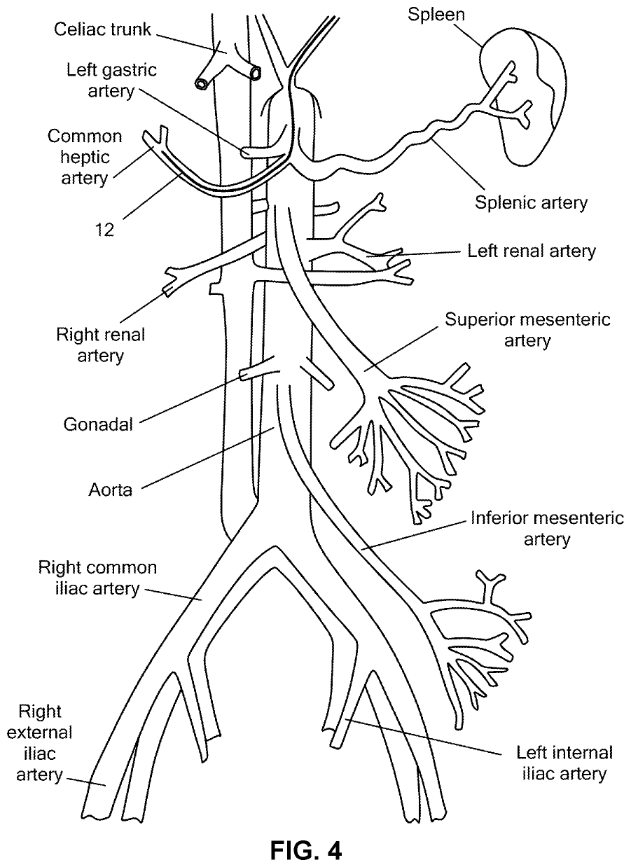

In one embodiment, as shown in more detail in FIG. 3, the glucose sensor 36 is a 3-electrode potentiostat configuration that includes a work electrode 38, a reference electrode 40, and a counter electrode 42 configured to run an electroanalytical analysis, in which the difference in electrode potentials is measured and studied. Although not described in detail herein, it is to be understood that the potentiostat generally is a circuit that allows isolation of the reference electrode 40 so no current flows through it, thereby preventing degradation of the reference electrode 40. The counter electrode 42 and reference electrode 40 are used together with the work electrode 38. The counter electrode 42 supplies electrons to the work electrode 38 for a peroxide oxidation reaction so the counter electrode 42 can run or control the electrodes. The counter electrode 42 serves as an amplifier that keeps the reference electrode 40 at a constant voltage so that the work electrode 38 will be run at a known voltage relative to the reference electrode. The counter electrode 42 may be driven at the same voltage as the reference electrode 40 with the counter amplifier.

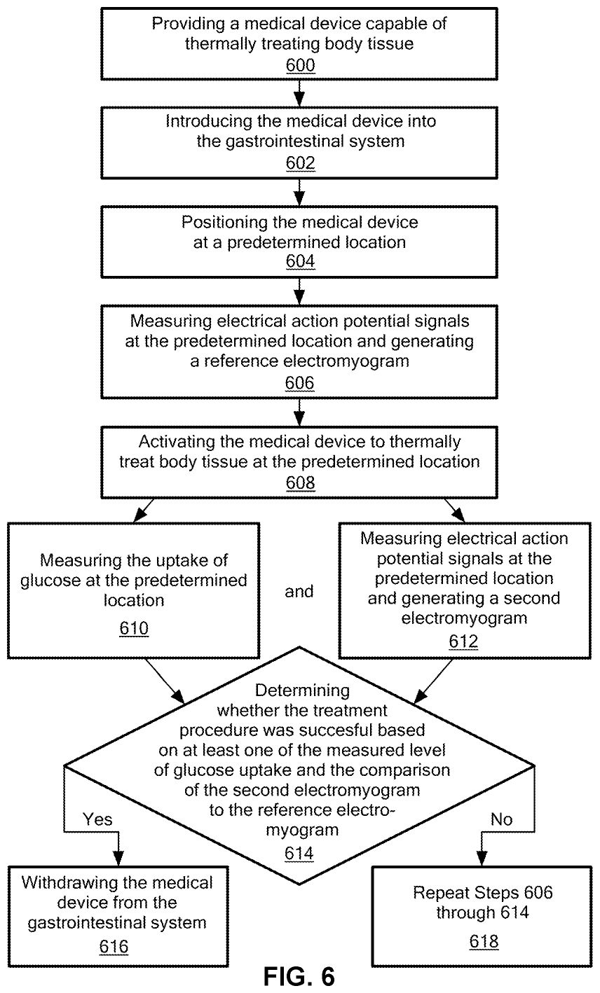

Now referring to FIG. 6, a method in accordance with the principles of the present disclosure described herein is shown. As mentioned above, the medical device 12 is first inserted within and passable through a patient's vasculature (Step 600) and navigated into the patient's gastrointestinal system (602) where the device 12 may then be positioned proximate to an area of target tissue within the hepatic artery for diagnosis or treatment (Step 604). Once the device 12 is in the desired position, the plurality of treatment electrodes 18 may be used to measure at least one electrical action potential signal (i.e., EMG recordings) at the area of target tissue and transmit the measured electrical action potential signals to the control unit 30 so that at least one of a reference electromyogram or baseline compound action potential (CAP) recording may be generated (Step 606). The generator 14 (either automatically or via manual input by the clinician) may then generate and deliver pulsed electric field (PEF) energy to the device 12, thereby activating the device 12, and specifically, the plurality of treatment electrodes 18 to thermally treat the area of target tissue (Step 608). Following, or during, the treatment procedure, the glucose sensor 36 positioned on the distal portion 24 of the elongate body 20, or proximate to the distal tip 26, may measure a level of glucose uptake at the area of target tissue to determine the efficacy of the treatment procedure (Step 610). Additionally, following the treatment procedure, the plurality of treatment electrodes 18 may be used to measure CAP signals and biological electrical activity or action potential signals (i.e., EMG recordings) at the area of target tissue and transmit the measured CAP signals and/or electrical action potential signals to the control unit 30 to generate a second electromyogram or second CAP recording (Step 612). The efficacy of the treatment procedure may then be determined based, in part, on at least one of the measured level of glucose uptake at the area of target tissue following the delivery of ablation energy, a comparison of the second generated electromyogram to the reference electromyogram, and a comparison of the second CAP recording to the baseline CAP recording (Step 614). When it is determined that the treatment procedure achieved the desired ablative effect in the area of target tissue, the device 12 may then be withdrawn from the gastrointestinal system (Step 616). If it is determined that the treatment procedure is not successful, the treatment procedure may be repeated and the device 12 would once again be activated to deliver PEF energy to the area of target tissue (Step 618).

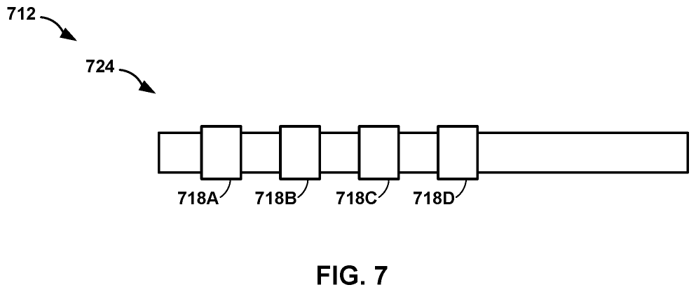

FIG. 7 is a conceptual diagram illustrating example energy vectoring, in accordance with one or more aspects of this disclosure. As shown in FIG. 7, device 712 may include distal portion 724 of an elongate body. Distal portion 724 may carry electrodes 718A-718D (collectively, “electrodes 718”). Device 712, distal portion 724, and electrodes 718 may respectively be considered examples of device 12, distal portion 24, and electrodes 18 of FIGS. 1 and 2.

In accordance with one or more aspects of this disclosure, a device may perform energy vectoring by controlling which of electrodes 718 are driven at which polarity (e.g., positive vs. negative). Examples of energy vectoring electrode configurations are shown below in Table 1 (the polarities of Table 1 are exemplary, and may be reversed). The device may perform energy vectoring in a unipolar or a bipolar configuration. Various electrode energy vectoring configurations may affect a depth and/or a width (e.g., a shape) of the ablation energy delivered via electrodes 718. In some examples, the device may adapt a depth and/or a width of the lesion based on pulse parameters and/or field distribution (e.g., with field distribution controlled via energy vectoring, which may be performed during Step 608 of FIG. 6). In this way, aspects of this disclosure enable more precise lesion shape control, which may improve treatment outcomes.

TABLE 1

| Positive | Negative |
|---|---|
| 718A | 718B, 718C, 718D |
| 718A, 718C | 718B, 718D |
| 718A, 718B, 718C | 718D |

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A system for hepatic nerve denervation, comprising:
a medical device, including:
an elongate body having a proximal portion and a distal portion opposite the proximal portion, the distal portion being configured to be in contact with an area of target tissue, the area of target tissue being an area of tissue within the hepatic artery; and
a plurality of treatment electrodes coupled to the distal portion; and
a generator in electrical communication with the medical device, the generator being configured to generate and deliver at least one pulse train of energy to the plurality of treatment electrodes to ablate the area of target tissue.

2. The system of claim 1, wherein the plurality of treatment of electrodes are arranged in a plurality of pairs, each pair of treatment electrodes having a polarity that is different than a polarity of an adjacent treatment electrode pair.

3. The system of claim 2, wherein the at least one pulse train of energy is delivered in one of a bipolar mode and a monopolar mode from the generator.

4. The system of claim 3, further including at least one sensing electrode coupled to the distal portion of the elongate body, the at least one sensing electrode being configured to continuously measure and record a plurality of glucose measurements within the area of target tissue, the plurality of glucose measurements being indicative of a measured level of glucose uptake within the area of target tissue.

5. The system of claim 4, further including:
a control unit in communication with the medical device; and
wherein at least one electrode pair of the plurality of treatment electrodes is configured to record at least one of an EMG signal and a compound action potential (CAP) signal and transmit the at least one of the EMG signal and CAP signal to the control unit.

6. The system of claim 5, wherein the control unit includes processing circuitry, the processing circuitry being configured to:
establish at least one of a reference electromyogram and baseline CAP recording of the area of target tissue prior to the delivery of the at least one pulse train of energy;
obtain at least one of an electromyogram and second CAP recording of the area of target tissue following the delivery of the at least one pulse train of energy;
at least one of:
compare the obtained electromyogram to the reference electromyogram; and
compare the second CAP recording to the baseline CAP recording; and
generate an alert based at least in part on the comparison.

7. The system of claim 6, wherein the processing circuitry is further configured to determine whether denervation has occurred in the area of target tissue based on the comparison of the obtained electromyogram and the reference electromyogram or the comparison of the second CAP recording to the baseline CAP recording.

8. The system of claim 7, wherein the control unit is further configured to discontinue the delivery of the at least one pulse train of energy from the generator when it is determined that denervation has occurred in the area of target tissue.

9. The system of claim 8, wherein the at least one pulse train of energy is delivered in a biphasic mode from the plurality of treatment electrodes.

10. The system of claim 4, wherein the medical device is further configured to determine whether denervation has occurred in the area of target tissue based on the measured level of glucose uptake within the area of target tissue.

11. A system for hepatic nerve denervation, comprising:
a medical device, including:
an elongate body having a proximal portion and a distal portion opposite the proximal portion, the distal portion being configured to be in contact with an area of target tissue, the area of target tissue being an area of tissue within the hepatic artery; and
a plurality of treatment electrodes coupled to and disposed about the distal portion; and
a generator in electrical communication with the medical device, the generator being configured to generate and deliver at least one pulse train of energy to the plurality of treatment electrodes to ablate the area of target tissue.

12. The system of claim 11, wherein the plurality of treatment of electrodes are arranged in a plurality of pairs, each pair of treatment electrodes having a polarity that is different than a polarity of an adjacent treatment electrode pair.

13. The system of claim 12, wherein the at least one pulse train of energy is delivered in one of a bipolar mode and a monopolar mode from the generator.

14. The system of claim 13, further including at least one sensing electrode coupled to the elongate body, the at least one sensing electrode being configured to continuously record a plurality of glucose measurements within the area of target tissue, the plurality of glucose measurements being indicative of a measured level of glucose uptake within the area of target tissue.

15. The system of claim 14, further including:
a control unit in communication with the medical device; and
wherein at least one electrode of the plurality of treatment electrodes is configured to record at least one of an EMG signal and a compound action potential (CAP) signal and transmit the at least one of the EMG signal and CAP signal to the control unit.

16. The system of claim 15, wherein the control unit includes processing circuitry, the processing circuitry being configured to:
establish at least one of a reference electromyogram and a baseline CAP recording of the area of target tissue prior to the delivery of the at least one pulse train of energy;
obtain at least one of an electromyogram and a second CAP recording of the area of target tissue following the delivery of the at least one pulse train of energy;
at least one of:
compare the obtained electromyogram to the reference electromyogram; and
compare the second CAP recording to the baseline CAP recording; and
generate an alert based at least in part on the comparison.

17. The system of claim 16, wherein the processing circuitry is further configured to determine whether denervation has occurred in the area of target tissue based on the comparison of the obtained electromyogram and the reference electromyogram or the comparison of the second CAP recording and the baseline CAP recording.

18. The system of claim 17, wherein the processing circuitry is further configured to discontinue the delivery of the at least one pulse train of energy from the generator when it is determined that denervation has occurred in the area of target tissue.

19. The system of claim 14, wherein the medical device is further configured to determine whether denervation has occurred in the area of target tissue based on the measured level of glucose uptake within the area of target tissue.

20. A method of denervating a hepatic nerve, the method comprising:
positioning a medical device proximate to an area of target tissue within the hepatic artery, the medical device including an elongate body with a proximal portion and a distal portion opposite the proximal portion;
measuring at least one EMG signal at the area of target tissue and generating a reference electromyogram, the at least one EMG signal being measured by a plurality of electrodes disposed on the distal portion of the elongate body;
delivering pulsed electric field (PEF) energy to plurality of electrodes to treat the area of target tissue, the delivered PEF energy being generated by an energy generator;
at least one selected from the group consisting of:
measuring a level of glucose uptake at the area of target tissue following the delivery of PEF energy; and
measuring at least one EMG signal at the area of target tissue following the delivery of PEF energy and generating a second electromyogram; and
determining an efficacy of the delivery of PEF energy based on at least one selected from the group consisting of the measured level of glucose uptake and a comparison of the second electromyogram to the reference electromyogram.
